(19) United States
(12) Patent Application Publication
(10) Pub. No.: US 2008/0215030 A1
(43) Pub. Date: Sep. 4, 2008

(54) Assembly for Filling a Container of a Delivery Device with a Pharmaceutical

(75) Inventor: Kenneth Alan Ritsher, Chicago, IL (US)

Correspondence Address: ELI LILLY & COMPANY, PATENT DIVISION, P.O. BOX 6288, INDIANAPOLIS, IN, 46206-6288, US

(73) Assignee: ELI LILLY AND COMPANY, Indianapolis, IN (US)

(21) Appl. No.: 11/997,205

(22) PCT Filed: Jul. 31, 2006
(86) PCT No.: PCT/US2006/029548

§ 371 Date: Jan. 29, 2008

Related U.S. Application Data
(60) Provisional application No. 60/706,333, filed on Aug. 8, 2005.

(51) Int. Cl. A61M 39/00

(52) U.S. Cl. 604/413

(57) ABSTRACT

A filling assembly for a container of a delivery device. The filling assembly includes a housing, a cartridge, and a transfer needle assembly. The housing is adapted to releaseably secure a fillable secondary container, and to movably mount the pharmaceutical filled cartridge. When the cartridge is advanced within the housing, a needle of the needle assembly pierces a septum of the fillable secondary container, as well as the cartridge septum. Advancement of the cartridge piston, such as with a lever equipped plunging tool of the filling assembly, forces pharmaceutical from the cartridge to the container through the needle.

### BACKGROUND OF THE INVENTION

The present invention pertains to medical devices, and, in particular, to an assembly for filling medicine into a container loadable into a delivery device.

A wide variety of medical devices are available which allow people, such as patients or health care professionals, to administer pharmaceuticals to themselves or others. Many of these devices are considered reusable but utilize a replaceable cartridge, which cartridge holds one or more doses of the desired pharmaceutical. While some existing cartridges, such as standard 3 ml glass cartridges, are a proven and readily available technology for packaging certain pharmaceuticals, unfortunately such cartridges are not an optimal size and shape for all devices. However, providing an optimally configured, prefilled container or cartridge for a given device is not always feasible, as such may require a substantial assembly line investment to achieve such filling, and further, depending on the materials of container construction, may raise regulatory issues associated with, for example, the long term stability of the pharmaceutical in such a prefilled container.

Thus, it would be desirable to provide an assembly for transferring or shuttling a pharmaceutical in a standard cartridge into a non-standard or less conventional container.

### BRIEF SUMMARY OF THE INVENTION

In one form thereof, the present invention provides a filling assembly including a housing, a cartridge, and a transfer needle assembly. The housing has a first end and a second end spaced in an axial direction, which first end is adapted to releaseably secure a fillable secondary container having a septum. The cartridge is mounted within the housing to be movable in the axial direction from a rearward position to a forward position, which cartridge includes a body, a movable piston and a septum, which body defines a pharmaceutical filled reservoir sealed at a rearward end by the movable piston and sealed at a forward end by the septum. The transfer needle assembly includes a cannula having a forward tip and a rearward tip, which transfer needle assembly is mounted within the housing to be movable in the axial direction from a first needle position to a second needle position by the cartridge moving from the rearward position to the forward position, wherein the transfer needle assembly is sized and configured with the housing to have a first arrangement when in the first needle position and a possible second arrangement when in the second needle position. The transfer needle assembly, when in the first needle position and in the first arrangement, blocks the cartridge from moving relative to the transfer needle assembly a distance sufficient to cause the rearward tip of the needle to pierce the cartridge septum, whereby the rearward tip is in non-piercing penetrating relationship with the cartridge septum, and further has the forward tip in non-piercing relationship with a septum of a fillable secondary container secured at the first end of housing. The transfer needle assembly, when in the second needle position and in the second arrangement, permits the cartridge to move relative to the transfer needle assembly during movement toward the forward position a distance sufficient to cause the rearward tip of the needle to pierce the cartridge septum and access the pharmaceutical filled reservoir, and has the forward tip in piercing relationship with the septum of a fillable secondary container secured at the first end of the housing for filling the container.

One advantage of the present invention is that a filling assembly may be provided which is simple and intuitive to use.

Another advantage of the present invention is that a filling assembly may be provided which uses a readily available, prefilled cartridge as the supply of pharmaceutical for a different container that requires filling.

Yet another advantage of the present invention is that a filling assembly may be provided having a plunging feature that utilizes a mechanical advantage to facilitate the manually operated process.

### BRIEF DESCRIPTION OF THE DRAWINGS

The above-mentioned and other advantages and objects of this invention, and the manner of attaining them, will become more apparent, and the invention itself will be better understood, by reference to the following description of embodiments of the invention taken in conjunction with the accompanying drawings, wherein:

FIG. 1 is a front perspective view, in exploded form, of an exemplary supply unit of a filling assembly of the present invention, as well as an empty container mountable to the unit for filling;

FIG. 2 is a front perspective view of the supply unit and fillable container of FIG. 1 in an assembled state;

FIG. 3 is a longitudinal cross-sectional view of the supply unit and fillable container of FIG. 2;

FIG. 4 is a front perspective view of an exemplary plunging tool of a filling assembly of the present invention, which tool is shown in an opened arrangement;

FIG. 5 is a front perspective view of the plunging tool of FIG. 4 in a closed arrangement;

FIG. 6 is a front perspective view of the plunging tool of FIG. 4 after being loaded with the supply unit and fillable container of FIG. 2;

FIG. 7 is a longitudinal cross-sectional view of the filling assembly and fillable container of FIG. 6;

FIG. 8 is a longitudinal cross-sectional view, similar to the view of FIG. 7, after the tool has been operated to advance a needle to pierce with its forward tip a filling septum of the fillable container;

FIG. 9 is a longitudinal cross-sectional view, similar to the view of FIG. 8, after the tool has been operated further to advance a supply cartridge such that a rearward tip of the needle has pierced a septum of that supply cartridge;

FIG. 10 is a longitudinal cross-sectional view, similar to the view of FIG. 9, after the tool has been operated further to force the pharmaceutical from the supply cartridge into the container;

FIG. 11 is a front perspective view of the supply unit and container of FIG. 10 after being removed as a unit from the plunger tool; and

FIG. 12 is a front perspective view of the supply unit and container of FIG. 11 after the filled container has been removed from the supply unit.

Corresponding reference characters indicate corresponding parts throughout the several views. Although the drawings represent an embodiment of the present invention, the drawings are not necessarily to scale, and certain features may be exaggerated or omitted in some of the drawings in order to better illustrate and explain the present invention.

### DETAILED DESCRIPTION OF THE INVENTION

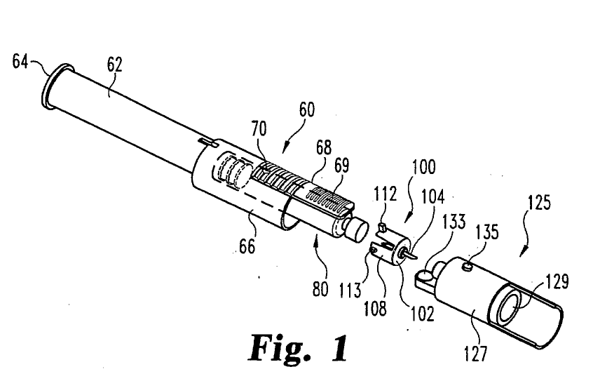

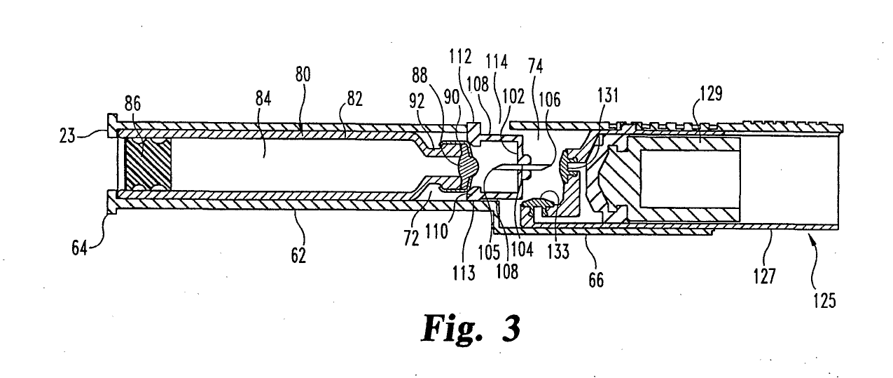

Referring initially to FIGS. 1-5, there is shown an exemplary embodiment of a filling assembly of the present invention. The filling assembly, generally designated 20, includes a plunging tool 30 and a pharmaceutical supply unit 60. Prior to its use, supply unit 60 holds the pharmaceutical product to be transferred into a fillable secondary container, such as a cartridge, with which the supply unit is configured to cooperate, and which container is shown at 125 in, for example, FIGS. 1-3. Plunging tool 30 serves to operate conveniently the supply unit to transfer the held product to the secondary container mounted to the supply unit.

For the shown embodiment, forward and rearward refer to relative locations on the filling assembly, wherein a direction of travel of the plunger during use to fill a container is considered a forward travel, and whereby an end of a plunger which contacts the piston of the supply cartridge to drive it forward to expel pharmaceutical from that cartridge is the plunger forward end. These references, as well as any other directional references in this detailed description with respect to the other Figures, such as inward or outward, or top or bottom, are intended for convenience of description, and by itself does not limit the present invention or any of its components to any particular positional or spatial orientation.

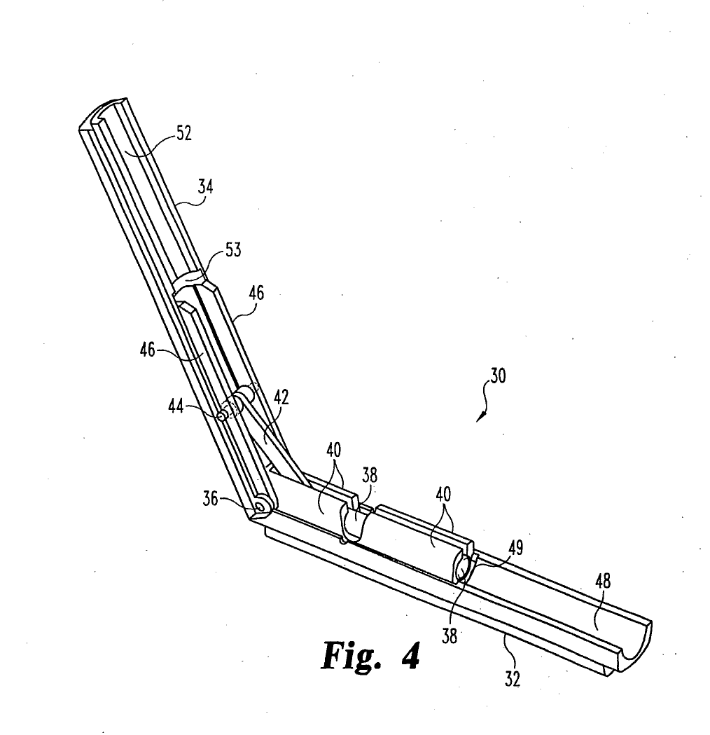

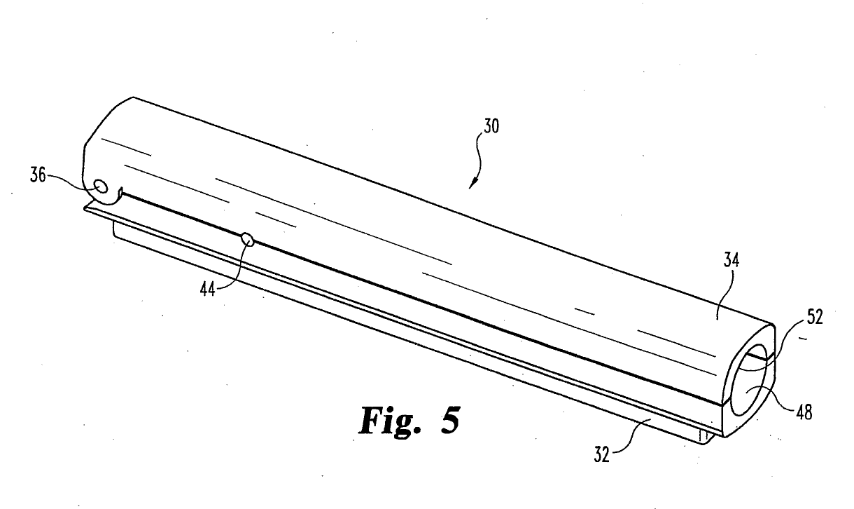

Plunging tool 30 includes a base 32 and an actuating lever 34 that are pivotally connected at 36. Tool 30 is shown in FIG. 4 in an open arrangement in which lever 34 has been manually pivoted away from base 32. In FIG. 5, tool 30 is shown closed with base 32 and lever 34 in their mating relationship. A rod-shaped plunger 38 is longitudinally slideable within a channel formed by upstanding guides 40 constructed integrally with the inward face of base 32. A connector bar 42 has one end pivotally pinned at 44 to flanges 46 of actuating lever 34, and an opposite end pivotally pinned to the rearward end region of plunger 38. Lever flanges 46 are sized to flank guides 40 when actuating lever 34 is shifted closed as described below.

Plunging tool 30 is cooperatively configured with supply unit 60 such that plunger 38 and at least a part of the supply unit housing move relative to each other during tool operation. In the shown embodiment, the forward region of the inward face of base 32 includes a longitudinally extending concavity 48 and a deeper, transverse groove 49. Groove 49 is located at the rearward end of concavity 48 and is formed in part by the forward end of guides 40. Concavity 48 and groove 49 are shaped to seat the exterior of a tubular housing portion 62 and lip 64, respectively, of supply unit 60. Lever 34 similarly includes a forward end with a concavity 52 and a groove 53 to accommodate housing portion 62 and lip 64 when lever 34 is fully operated. Other complementary constructions of the plunging tool and the supply unit to maintain portions of such pieces in a proper operational arrangement may be employed within the scope of the invention. As plunging tool 30 is intended to be reusable with additional supply units, tool 30 is made out of one or more durable materials, such as metal or sturdy plastics.

The external housing of supply unit 60 may be made in one piece of plastic. The housing includes the cylindrical tubular portion 62, the circumferential lip 64 at the rearward end of portion 62, and another cylindrical tubular portion 66 with a tab 68 at the forward end of housing portion 62. Housing portion 66 has a diameter that is larger than that of tubular portion 62. A hole 70 in tab 68 is disposed rearward of a gripping region 69 of tab 68 which projects forward of tubular housing portion 66.

An interior hollow 72 of housing portion 62 is open at the rearward end of housing portion 62 to allow insertion of plunger 38. Hollow 72 opens to an interior hollow 74 of housing portion 66. Hollow 72 is sized to receive a pharmaceutical-filled container, generally designated 80, that is inserted therein axially through hollow 74 during manufacturing assembly. A circumferential rib 73 of housing portion 62 that radially inwardly projects into hollow 72 at its rearward end is sized to prevent rearward removal of container 80 from hollow 72.

Container 80 is a cartridge of a conventional size and shape, such as a 3 ml cartridge, and includes a barrel 82 made of glass, a pharmaceutical-filled reservoir 84 within the barrel, a piston 86, a septum 88 and a cap 90. Piston 86 seals the rearward end of reservoir 84 and is axially slidably and sealably engaged with the barrel interior wall. Septum 88 is held by a cap 90 that is secured to a stepped-down diameter neck portion 92 of the barrel, and septum 88 seals the forward end of reservoir 84.

Forward of cartridge 80, a transfer needle assembly, generally designated 100, is disposed within the interior of the supply unit housing to be longitudinally shiftable therein. Assembly 100 includes a plastic base 102 that axially retains a metal cannula or needle 104 having a rearward point 105 and a forward point 106. Rearwardly extending from and integrally molded with base 102 are at least two, such as four equally angularly spaced, resilient fingers 108. The radially inward face of a rearward region of each resilient finger 108 includes a ramped shoulder 110 located rearward of needle tip 105. Shoulder 110 is shaped and sized to be directly abutted by cartridge cap 90 for a camming outward of fingers 108 as described further below.

One finger 108 also includes a radially extending tab 112 sized to fit and slide within a longitudinally extending slot 114 formed in both the forward end of housing portion 62 and the rearward end of housing portion 66. The other fingers 108 include ribs 113 for abutting the surface defining hollow 72. During manufacturing assembly, and after cartridge 80 has been inserted in hollow 72, needle assembly 100 is inserted axially through hollow 74 such that tab 112 enters slot 114, and needle assembly insertion continues until tab 112 reaches the rearward end of slot 114.

When needle assembly 100 is so inserted, fingers are closely backed up by the hollow-forming surface of housing portion 62, and shoulders 110 are too close together to allow cartridge cap 90 to enter the space between fingers 108 where needle tip 105 resides. In contrast, when tab 112 is located at the forward end of slot 114, whereat the fingers 108 are not closely backed up by the interior surface defining hollow 74, or for that matter any other part of the device, fingers 108 can be splayed open by cap 90 pushing shoulders 110 outward to allow cartridge cap 90 to enter the space between fingers 108.

Hollow 74 is sized and shaped to insertably receive the secondary container, generally designated 125, to be filled by use of filling assembly 20. Supply unit 60 is also adapted to releasably secure container 125 when mounted together. In the shown embodiment, the secondary container is an empty, disposable cartridge unit that can be preassembled to supply unit 60 by the manufacturer. Container 125 is shown including a plastic body 127, an axially movable piston 129, shown with a rigid core and a resilient sealing end, that closes off one end of the medicine-fillable reservoir of the cartridge 125, a filling needle-pierceable septum 131 that covers a body opening to serve as filling port of the container, and a dispensing needle-pierceable septum 133 that covers an opening in a protuberance of the body to serves as a dispensing port of the container when loaded into an appropriate delivery device. Container 125, which alternatively may be assembled from other differently configured pieces, including a glass liner, will be further, understood in view of U.S. Provisional Patent Application No. 60/591,570 and International Patent Application No. PCT/US05/010580, the complete disclosures of which applications are hereby incorporated herein by reference.

To facilitate being secured with the shown supply unit 60, container 125 also includes a connecting module in the form of a detent 135 formed integrally with and projecting from body 127. Detent 135 is positioned on body 127 to fit within tab hole 70 when container 125 is probably oriented and inserted into supply unit 60, such as by the manufacturer, which fit hinders withdrawal of the container from the supply unit until a user grips the gripping region 69 and lifts tab 68 away from the rest of the housing portion 66 to release the detent/hole fit. Tab 68 is scored or otherwise formed to be rendered not reuasable after being lifted or pulled from the supply unit housing to release detent 135.

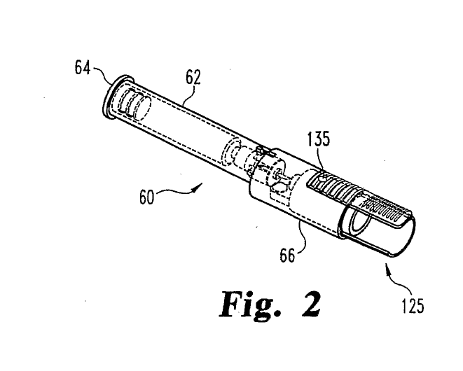

The structure of filling assembly 20 will be further understood in view of the following description of a use thereof. A user of a delivery device who requires a filled container 125 for insertion into that device would obtain a pharmaceutical supply unit 60 arranged as shown in FIG. 2. Although the supply unit would likely already be loaded with the desired, unfilled container 125, a user could initially mount to the supply unit an unused and unfilled container for filling.

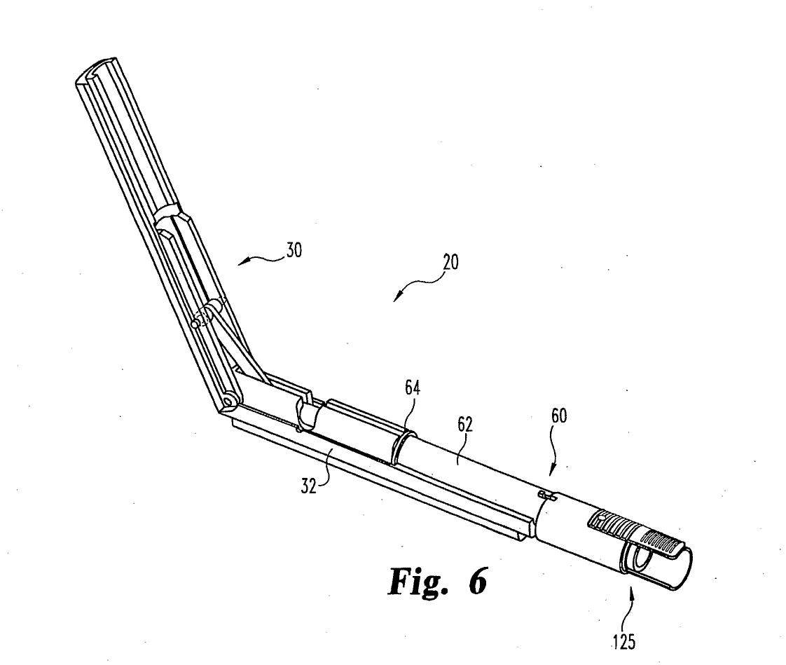

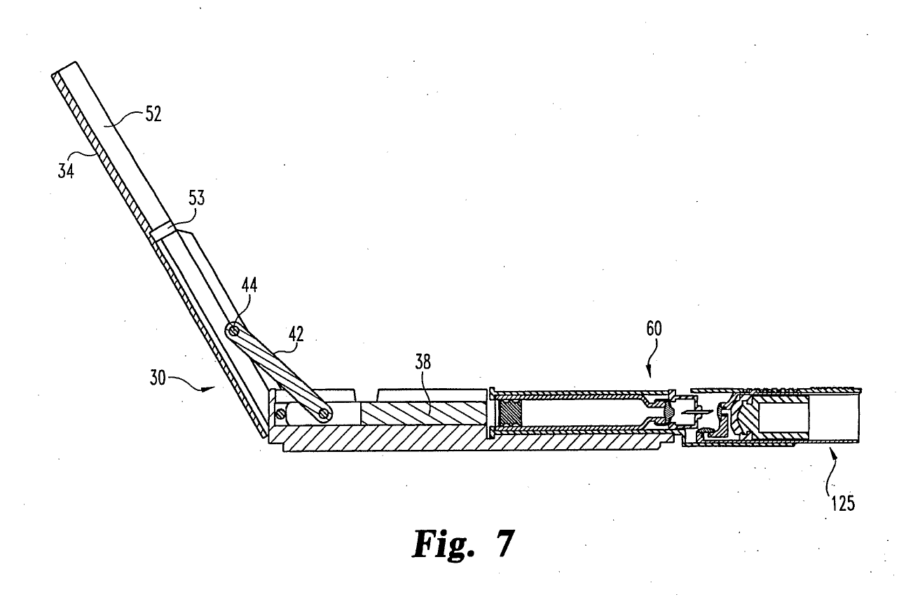

The supply unit then can be operated by inserting a slender object, such as a pencil, through the rearward end of the supply unit housing to abut and drive cartridge piston 86 in a manner more fully described below. Typically, however, supply unit 60 will be loaded into plunging tool 30 by the user pivoting lever 34 away from base 32 to the arrangement shown in FIG. 4, orienting and aligning supply unit 60 and tool 30, and then placing the supply unit such that tubular housing portion 62 nests in concavity 48 with lip 64 fitting within groove 49, thereby axially locating the supply unit 60 within the plunging tool 30. At this point, filling assembly 20 is arranged as shown in FIGS. 6 and 7.

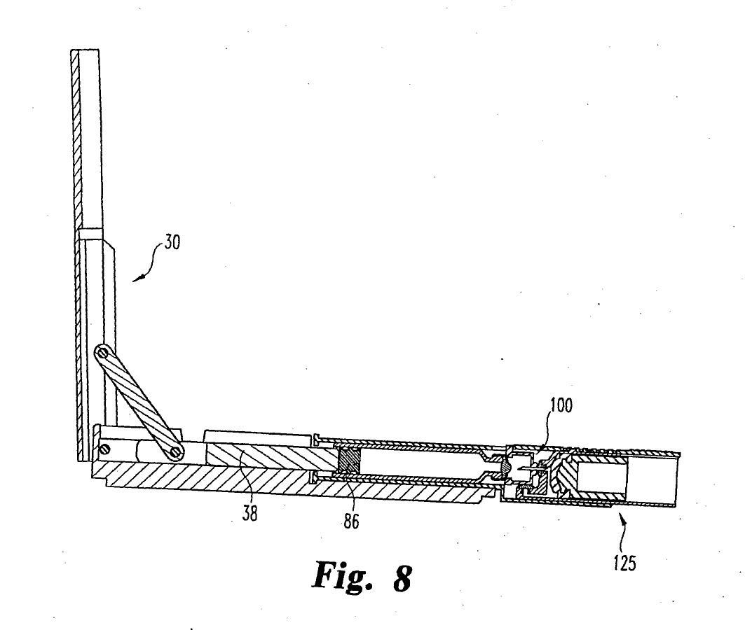

Next, the user manually pivots lever 34 downward toward base 32 and the nested supply unit 60, such that plunger 38 is driven forward. As plunger 38 so moves forward, the forward end of the plunger initially enters interior hollow 72 through the opened rearward end of housing portion 62, and continues forward until abutting cartridge piston 86. Additional forward movement of plunger 38 thereby starts to advance the entire cartridge 80, as the supply of fluid within the cartridge essentially acts as an incompressible object as the cartridge septum 88 prevents fluid flow from the reservoir. As cartridge 80 so advances, and after closing up, if present, any axial gap with shoulders 110 of needle assembly 100, cartridge cap 90 abuts shoulders 110, and continued advancement of the cartridge shifts transfer needle assembly 100 axially forward until the forward point 106 of needle 104 penetrates septum 131 of secondary container 125. Forward motion of the transfer needle assembly 100 is halted when tab 112 abuts the forward edge of slot 114. At this point, filling assembly 20 is arranged as shown in FIG. 8.

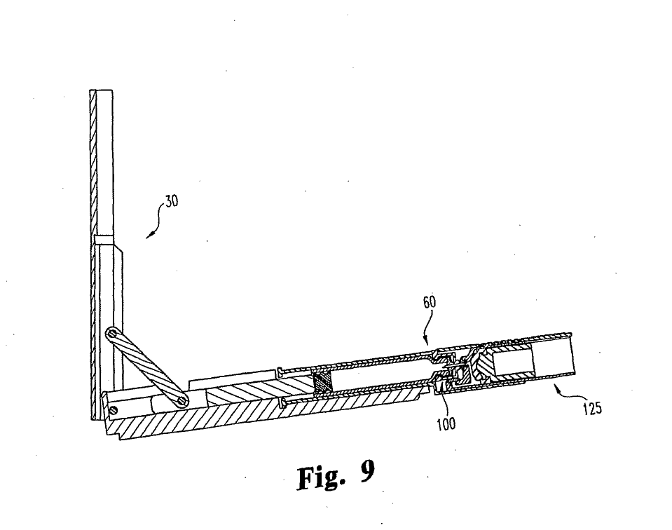

At this arrangement, fingers 108 are axially positioned clear of housing portion 62 so as to be splayable open. As the cartridge continues to move forward by further pivoting of lever 34, shoulders 110 are abutted by cap 90 to cam fingers 118 open to allow the receipt of cartridge cap 90. During the cap receipt, the rearward point 105 of the now halted needle assembly 100 has its needle tip 105 pass through a central opening in the forward end of cap 90 and pierce cartridge septum 88 to provide a fluid flow outlet by which medicine within reservoir 84 can be transferred through tip 105, the length of needle 104, and forward point 106 into secondary container 125. When shoulders 110 have been passed by cap 90, fingers 108 snap inward to lock and needle assembly 100 to cartridge cap 90. At this point, filling assembly 20 is arranged as shown in FIG. 9.

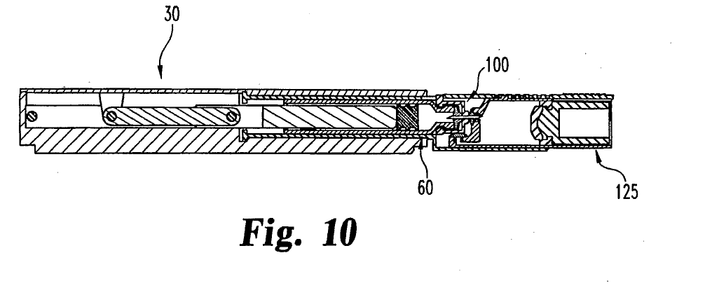

Plunger 38 is sized in conjunction with the other components of the filling assembly 20 such that when lever 34 has been pivoted closed so as to abut the base 32, at which point the plunger unit cannot be closed further, plunger 38 has been driven forward enough to sufficiently empty the contents of cartridge reservoir 84 into secondary container 125, whereby the container piston 129 has moved forward as the medicine filled the reservoir of that container has filled. At this time, the filling assembly 20 and secondary container 125 are arranged as shown in FIG. 10.

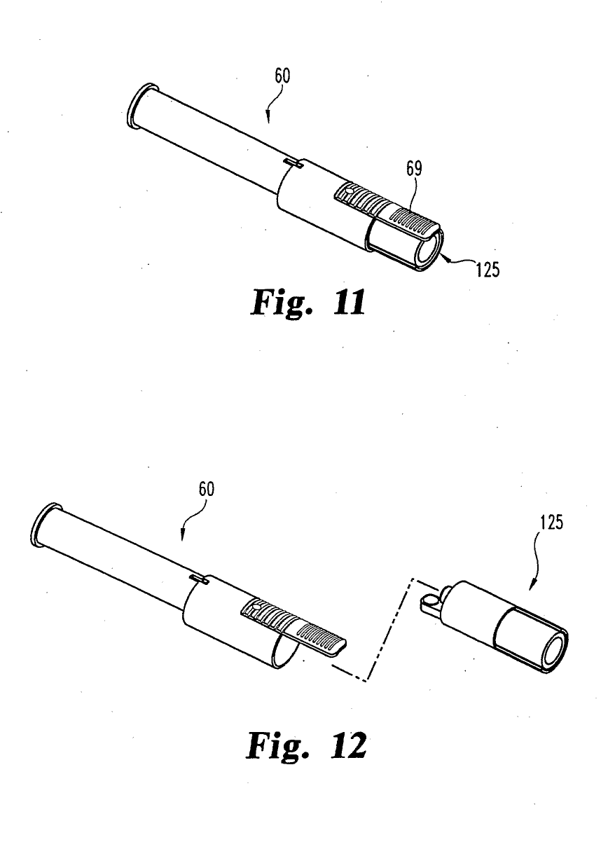

Transfer needle 104 remains fully enclosed within the supply unit housing, as it is secured with cap 90. After supply unit 60 is removed from plunging tool 30 by pivoting upward lever 34 and simply lifting supply unit 60 from its nested arrangement with the base, the supply unit 60 and container 125 are arranged as shown in FIG. 11. Then, by gripping region 69 and pulling upward, a user can tear or lift clear the tab 68 containing hole 70 so as to free detent 135, such that the filled secondary container 125 can be removed from supply unit 60, as shown in FIG. 12. Supply unit 60 can be discarded in the normal course, and plunger tool 30 can be stored for subsequent use with a replacement supply unit 60.

The filled secondary container 125 then can be inserted into the intended pharmaceutical delivery device, such as the one disclosed in the International Patent Application Number PCT/US05/010580, for an injection delivery of the pharmaceutical in a desired fashion.

While this invention has been shown and described as having preferred designs, the present invention may be modified within the spirit and scope of this disclosure. For example, the supply unit can be adapted to mount and fill containers of other designs, including compressible containers or bladders or the like, or a container having a slidable piston that includes a septum through which cartridge filling may occur. This application is therefore intended to cover any variations, uses or adaptations of the invention using its general principles. Further, this application is intended to cover such departures from the present disclosure as come within known or customary practice in the art to which this invention pertains.

We claim:

1. A filling assembly comprising:
a housing having a first end and a second end spaced in an axial direction, said first end adapted to releaseably secure a fillable secondary container having a septum;
a cartridge mounted within said housing to be movable in said axial direction from a rearward position to a forward position, said cartridge including a body, a movable piston and a septum, said body defining a pharmaceutical filled reservoir sealed at a rearward end by said movable piston and sealed at a forward end by said septum;
a transfer needle assembly including a cannula having a forward tip and a rearward tip, said transfer needle assembly mounted within said housing to be movable in said axial direction from a first needle position to a second needle position by said cartridge moving from said rearward position to said forward position, wherein said transfer needle assembly is sized and configured with said housing to have a first arrangement when in said first needle position and a possible second arrangement when in said second needle position;
wherein said transfer needle assembly, when in said first needle position and in said first arrangement, blocks said cartridge from moving relative to said transfer needle assembly a distance sufficient to cause said rearward tip of said needle to pierce said cartridge septum, whereby said rearward tip is in non-piercing penetrating relationship with the cartridge septum, and further has said forward tip in non-piercing relationship with a septum of a fillable secondary container secured at the first end of said housing; and
wherein said transfer needle assembly, when in said second needle position and in said second arrangement, permits said cartridge to move relative to said transfer needle assembly during movement toward said forward position a distance sufficient to cause said rearward tip of said needle to pierce said cartridge septum and access said pharmaceutical filled reservoir, and has said forward tip in piercing relationship with the septum of a fillable secondary container secured at the first end of said housing for filling the container.

2. The filling assembly of claim 1 wherein said transfer needle assembly includes a needle supporting base and at least one resilient finger extending rearwardly beyond said rearward tip for blocking said cartridge when said transfer needle assembly is in said first needle position and in said first arrangement.

3. The filling assembly of claim 1 wherein said transfer needle assembly includes a projecting tab that engages a complementary opening in said housing to locate said transfer needle assembly.

4. The filling assembly of claim 1 wherein said housing comprises a connecting module structured and arranged to engage a complementary connecting module of the secondary container to secure the container to said housing.

5. The filling assembly of claim 1 wherein said housing comprises a pullable tab, and said connecting module of said housing comprises an opening in said tab.

6. The filling assembly of claim 1 further comprising a plunging tool including a base and a plunger, said base complementarily configured with said housing to axially retain said housing relative to said base, wherein said plunger fits within said cartridge to drivingly engage said cartridge piston, and wherein said plunger and said base are axially movable relative to each other during plunging tool operation.

7. The filling assembly of claim 6 wherein said plunging tool comprises a lever pivotally connected to said base and connected to said plunger such that pivoting of said lever relative to said base drives said plunger into said housing.

8. The filling assembly of claim 6 wherein said housing comprises a cylindrical exterior that nests within a concavity of said base.
